(19) United States
(12) Plant Patent Application Publication
(10) Pub. No.: US 2019/0357402 P1
(43) Pub. Date: Nov. 21, 2019

(54) Phalaenopsis orchid plant named 'PHALFOSFLY'

(50) ;

(71) Applicant: ANTHURA B.V., Bleiswijk (NL)

(72) Inventor: Martinus Nicolaas Gerardus Van SWIETEN, Utrecht (NL)

(21) Appl. No.: 15/932,897

(22) Filed: May 17, 2018

(51) Int. Cl. A01H 6/62

(52) U.S. Cl. PLT/311

(57) ABSTRACT

A new and distinct variety of Phalaenopsis plant named ‘PHALFOSFLY’, particularly characterized by having very light purple flowers with a darker purple shade and a purple lip, 1 to 3 peduncles that are medium long and moderate, leaves that are narrow obovate, and is propagated by meristem tissue culture, is disclosed.

Genus and species: Phalaenopsis hybrid.

Variety denomination: ‘PHALFOSFLY’.

### BACKGROUND OF THE NEW PLANT

The present invention relates to a new and distinct cultivar of Phalaenopsis plant, botanically known as Phalaenopsis hybrid of the Orchidaceae family, commonly referred to as moth orchid, and hereinafter referred to by the variety name ‘PHALFOSFLY’.

The new Phalaenopsis plant is a product of a planned breeding program conducted by the inventor in Bleiswijk, The Netherlands. The objective of this breeding program was to create a new Phalaenopsis plant with numerous attractive and unique, very light purple flowers with a darker purple shade and a purple lip, suitable for potted plant production.

The new Phalaenopsis plant ‘PHALFOSFLY’ is a result of cross-pollination made by the inventor in November 2007 in Bleiswijk, The Netherlands of the proprietary female, or seed parent, Phalaenopsis hybrid ‘21062-01’ (unpatented) with the proprietary male, or pollen parent, Phalaenopsis hybrid ‘08667-02’ (unpatented).

The new Phalaenopsis was selected by the inventor as a single plant within the progeny of the stated cross-pollination in a controlled greenhouse in Bleiswijk, The Netherlands in November 2010. Asexual reproduction of the new Phalaenopsis plant by meristem tissue culture since 2013 in Bleiswijk, The Netherlands, has demonstrated that the new variety reproduces true to type with all of the characteristics, as herein described, firmly fixed and retained through successive generations.

Plant Breeder's Rights for this variety have been applied for in Europe on Apr. 25, 2017. ‘PHALFOSFLY’ has not been made publicly available or sold anywhere in the world more than one year prior to the effective filing date of this application.

### SUMMARY OF THE INVENTION

The following are the most outstanding and distinguishing characteristics of this new cultivar when grown under normal horticultural practices in Bleiswijk, The Netherlands and can be used to distinguish ‘PHALFOSFLY’ as a new and distinct variety of Phalaenopsis plant.
1) Very light purple flowers with a darker purple shade and a purple lip;
2) 1 to 3 peduncles;
3) Peduncle is medium long and moderate; and
4) Shape of the leaf is narrow obovate.

### DESCRIPTION OF THE PHOTOGRAPHS

This new Phalaenopsis plant is illustrated by the accompanying photographs which show the overall plant habit including blooms, buds and foliage of the plant; the colors shown are as true as can be reasonably obtained by conventional photographic procedures. The photographs were taken in a greenhouse in Bleiswijk, The Netherlands, from 50-week old plants in March 2018. Colors in the photographs may differ from the color values cited in the detailed botanical description, which accurately describe the actual colors of the new variety.

FIG. 1 shows the overall plant habit, including blooms, buds and foliage of ‘PHALFOSFLY’.

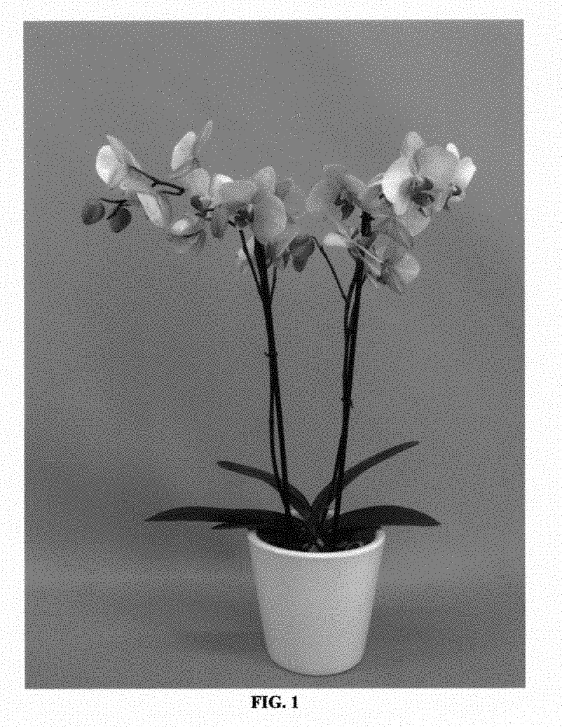

FIG. 2 shows a close-up of a flower of ‘PHALFOSFLY’.

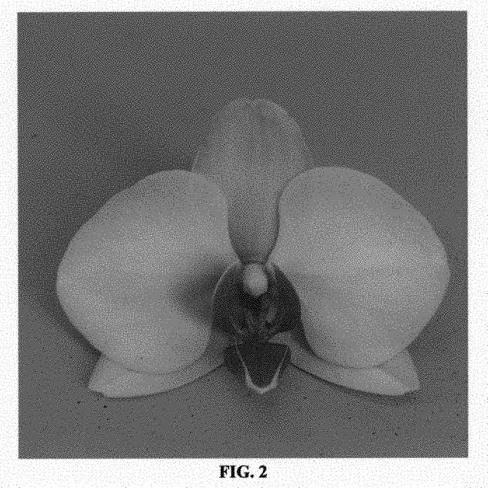

FIG. 3 shows an overhead view of the leaves of ‘PHALFOSFLY’.

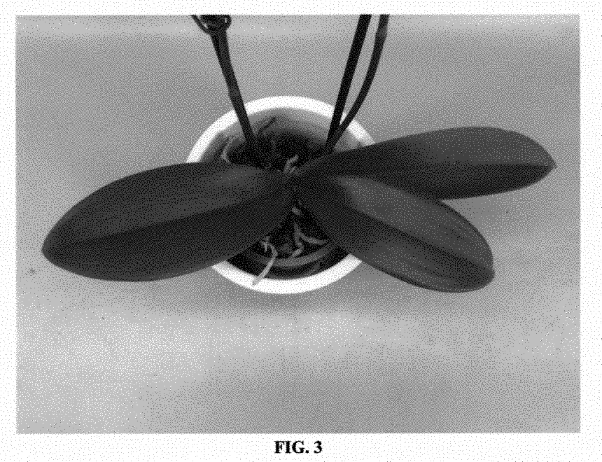

### DESCRIPTION OF THE NEW VARIETY

The following detailed description sets forth the distinctive characteristics of ‘PHALFOSFLY’. Plants of the new Phalaenopsis have not been observed under all possible environmental conditions. The phenotype may vary somewhat with variations in environment such as temperature, light intensity and day length, without, however, any variance in genotype. The chart used in the identification of colors described herein is the R.H.S. Colour Chart of The Royal Horticultural Society, London, England, 2015 edition, except where general color terms of ordinary significance are used. The color values were determined under 4000-6000 lux natural light in a greenhouse in Bleiswijk, The Netherlands. Observations and measurements were made in March 2018 on flowering plants which were planted in 12 centimeter (diameter) pots. After in-vitro propagation, the plants were grown in nursery trays for 20-24 weeks, followed by transplantation to 12 centimeter (diameter) pots and grown in a greenhouse between 27° C. to 29° C. for 30 weeks, continued by a cooling period of 8 weeks between 18° C. to 20° C. and 12 weeks in a greenhouse of 21° C. Flowering occurs after 50-weeks in a 12 centimeter pot.

### DETAILED BOTANICAL DESCRIPTION

Classification:
   Family.—Orchidaceae.
   Botanical.—Phalaenopsis hybrid.
   Common name.—Moth orchid.
   Variety name.—‘PHALFOSFLY’.
Parentage:
   Female parent.—Phalaenopsis cultivar ‘21062-01’ (unpatented).
   Male parent.—Phalaenopsis cultivar ‘08667-02’ (unpatented).
Propagation:
   Type.—Meristem tissue culture.
Roots:
   Root description.—Greyed-green colored roots (RHS 190B/C) with branching lateral roots having light green (RHS 145D) colored root tips.
Plant:
   Commercial crop time to flowering.—Following asexual propagation (in-vitro), the rooted cuttings grow for 20-24 weeks. After transplantation into 12 cm pots, the plants are finished after 48 to 50 weeks.
   Growth habit of peduncle.—Standard, green leaves, panicle.
   Height (from soil level to top of inflorescence).—Approximately 46.0 cm to 51.0 cm.
   Width (measured from leaf tips).—About 32.0 cm to 34.0 cm.
   Vigor.—Moderate.
Leaves:
   Mature leaves.—Quantity per plant: 6 to 9 leaves are produced before flowering. Length (fully expanded): 15.0 cm to 18.0 cm. Width: 5.5 cm to 6.5 cm. Shape: Obovate. Base shape: Moderately elongated. Apex: Obtuse unequal. Leaf blade angle with the petiole (measured from the horizontal position): Between 10 degrees and 25 degrees. Leaf margin: Entire. Color: Upper surface: RHS 146A. Lower surface: RHS 146B with brown (RHS 200C) margin. Texture: Rough. Thickness: 2.5 mm to 3.0 mm. Venation: Pattern: Parallel. Color of the midvein: Upper surface: RHS 146A. Lower surface: RHS 146B.
Peduncle:
   Quantity per plant.—1 to 3.
   Number of flowers per peduncle.—16 to 24.
   Length.—46.0 cm to 51.0 cm.
   Diameter.—4.0 mm to 4.5 mm.
   Strength.—Moderate.
   Aspect.—Upright to slightly pendant.
   Texture.—Smooth.
   Color.—Mix of brown (RHS 200A) and light green (RHS 146B).
   Internode length.—3.5 cm to 4.5 cm.
   Callosities.—None.
Inflorescence description:
   Appearance.—Upright to slightly pendant, panicle inflorescence with bilaterally symmetrical flowers that open in succession beginning with the lowermost flower.
   Inflorescence size.—Height (from base to tip): 220.0 mm to 260.0 mm.
   Flowering time.—First flowers can be expected 10 to 11 months after planting in a 12 cm (diameter) pot.
   Flower.—Height: 67.0 mm to 72.0 mm. Diameter: 77.0 mm to 82.0 mm. Depth of lip: 18.0 mm to 21.0 mm.
   Flower longevity.—On the plant: 8 to 12 weeks.
   Fragrance.—Absent.
   Flower bud.—Average size: Large. Length: 23.0 mm to 25.0 mm. Width: 20.0 mm to 22.0 mm. Shape: Egg shaped. Color: Light purple (RHS 77C) with a touch of light green (RHS 145D) and red (RHS 182B).
   Petals.—Arrangement: Open/free. Shape: Semi-circular. Apex: Obtuse asymmetric. Margin: Slightly undulated. Length (from base to tip): 36.0 mm to 38.0 mm. Width: 42.0 mm to 44 0 mm. Color (when fully opened): Upper surface: Basic color: Pinkish-white (RHS NN155B). Over color: Light purple (RHS 76C). Lower surface: Basic color: Very light purple (RHS 76D). Over color: Light purple (RHS 76B).
   Dorsal sepal.—Shape: Elliptic. Apex: Emarginated symmetric. Margin: Entire. Length (from base to tip): 38.0 mm to 40.0 mm. Width: 27.0 mm to 29.0 mm. Color (when fully opened): Upper surface: Basic color: Purple (RHS N75C). Over color: Light purple (RHS 76C). Lower surface: Basic color: Purple (RHS N75C). Over color: Very light green (RHS 145D) in the middle.
   Lateral sepals.—Shape: Ovate. Apex: Acute asymmetric. Margin: Entire. Length (from base to tip): 39.0 mm to 41.0 mm. Width: 22.0 mm to 24.0 mm. Color (when fully opened): Upper surface: Basic color: Very light purple (RHS 76D). Over color: Light purple (RHS 76B) toward the margin. Lower surface: Basic color: Purple (RHS N75C). Over color: Light green (RHS 145C/D) at the base with a red-purple (RHS N78B) vein.
   Labellum (lip).—Whiskers: Present. Length of whiskers: 13.0 mm to 15.0 mm. Color of whiskers: Light purple (RHS 76A/B) and lighter purple (RHS 76C) toward the tip. Pubescence on the lip: Absent.
   Lateral lobe.—Shape: Type IV (as described in the International Union for the Protection of New Varieties of Plants (UPOV) Test Guidelines for Phalaenopsis); weakly spatulate. Margin: Slightly undulated. Length: 17.0 mm to 19.0 mm. Width: 10.0 mm to 12.0 mm. Color: Yellow (RHS 6C/D) with dark red (RHS 59A) spots at the base; dark red (RHS 59A) and red-purple (RHS N78B) toward the margin.
   Apical lobe.—Shape: Triangular. Margin: Entire. Length: 20.0 mm to 22.0 mm. Width: 14.0 mm to 16.0 mm. Color: Red (RHS 59B) at the base; red-purple (RHS N78B) toward the whiskers.
   Callus.—Average size: Small to medium. Height: 5.0mm to 6.0 mm. Length: 4.0 mm to 5.0 mm. Width: 3.0 mm to 4.0 mm. Color: Yellow (RHS 9A) dotted (RHS 59A).
   Reproductive organs.—Column: Length: 9.0 mm to 11.0 mm. Diameter: 4.0 mm to 4.5 mm. Color: White (RHS NN155C). Pollinia: Quantity: 2. Diameter: 1.1 mm to 1.4 mm. Color: Orange (RHS 26A). Ovary: Length: 10.0 mm to 12.0 mm. Diameter: 2.1 mm to 2.3 mm. Pedicel: Length: 35.0 mm to 37.0 mm. Diameter: 2.4 mm to 2.6 mm. Color: Dark red (RHS 183B) at the base; light green (RHS 146D) and light purple (RHS N77B) toward the flower.
Disease, pest, and stress resistance: No specific resistance or susceptibility observed to pathogens and pests common to Phalaenopsis to date.
Fruit and seeds: Fruit and seed development has not been observed on plants of the new Phalaenopsis to date.

### COMPARISON WITH PARENTAL LINES AND MOST SIMILAR VARIETIES

‘PHALFOSFLY’ differs from female parent plant ‘21062-01’ (unpatented) in that ‘PHALSOSFLY’ has light purple, flowers with a shaded pattern, an obtuse petal apex, an emarginated dorsal sepal apex, apical lobes with red at the base and reddish purple toward the whiskers, and light purple whiskers, whereas ‘21062-01’ has white flowers with a flecked pattern, an emarginated petal apex, an obtuse dorsal sepal apex, apical lobes with a brown margin toward the callus, yellow at the base and white toward the whiskers, and white whiskers. Additionally, ‘PHALFOSFLY’ has smaller flowers than ‘21062-01’.

‘PHALFOSFLY’ differs from male parent plant ‘08667-02’ (unpatented) in that ‘PHALFOSFLY’ has flowers with a shaded pattern and light purple whiskers, whereas ‘08667-02’ has flowers with a shaded and striped pattern and dark red whiskers. Additionally ‘PHALFOSFLY’ has smaller flowers than ‘08667-02’.

‘PHALFOSFLY’ is most similar to the commercial Phalaenopsis plants named ‘PHALDILEP’ (U.S. Plant patent application Ser. No. 15/530,069) and ‘PHALBANEHI’ (unpatented). ‘PHALFOSFLY’ differs from the commercial variety ‘PHALDILEP’ in that ‘PHALFOSFLY’ has light purple whiskers, whereas ‘PHALDILEP’ has white whiskers. Additionally, ‘PHALFOSFLY’ has narrower petals and dorsal sepals than ‘PHALDILEP’.

‘PHALFOSFLY’ differs from the commercial variety ‘PHALBANEHI’ in that ‘PHALFOSFLY’ has a triangular apical lobe shape, a white column, and light purple whiskers, whereas ‘PHALBANEHI’ has a rhombic apical lobe shape, a light red-purple column, and white whiskers. Additionally, ‘PHALFOSFLY’ has narrower petals and dorsal sepals than ‘PHALBANEHI’.

I claim:

1. A new and distinct variety of Phalaenopsis plant named ‘PHALFOSFLY’, substantially as described and illustrated herein.
